(19) United States
(12) Patent Application Publication
(10) Pub. No.: US 2006/0280694 A1
(43) Pub. Date: Dec. 14, 2006

(54) Composition for the mineralization of dental hard tissues and the reduction of caries-inducive microflora

(76) Inventors: John Peldyak, Mt. Pleasant, MI (US); Kauko Makinen, Uusikaupunki (FI); Larry Bybee, Pocatello, ID (US); Russ Misner, Pocatello, ID (US)

Correspondence Address: Hillary J. Morgan;Attorney at Law, 890 S. 500 E., Orem, UT, 84097, US

(21) Appl. No.: 11/148,653

(22) Filed: Jun. 9, 2005

(51) Int. Cl. A61K 8/34; A61K 8/73; A61K 8/19

(52) U.S. Cl. 424/49

(57) ABSTRACT

The present invention relates to a composition having increased levels of minerals for mineralizing hard tissues within the oral cavity and an ability to reduce caries-inducive microflora. The composition also has, among other things, an anti-dental caries function that promotes beneficial oral health.

### BACKGROUND OF THE INVENTION

1. The Field of the Invention

The present invention is in the field of oral compositions. More particularly, the invention relates to an oral composition that mineralizes dental hard tissues in the oral cavity and attempts to reduce caries-inducive microflora.

2. The Relevant Technology

Dental caries is one of the most prevalent human diseases existing today, affecting all ages and demographics. Dental caries is generally caused by the interaction of the following three factors: a susceptible tooth surface, proper oral microflora, and suitable substrates for the microflora. Dental caries is normally initiated and characterized by the demineralization of mineral components and the decomposition of the organic matrix from one or more calcified structures of a tooth: enamel, dentin, or cementum

A normal tooth surface is covered by enamel, which is a protective surface layer covering dentin. Enamel, the hardest structure of a tooth, is composed of calcium, phosphate, and other ions in a hydroxyapatite-like structure. Root surfaces of a tooth are covered by cementum, which is a thin protective surface layer.

Acidity within the oral cavity is generally caused by either foods introduced into the oral cavity or by bacteria. Some bacteria that exist in biofilm or throughout the oral cavity may create harmful acids by consuming fermentable carbohydrates. As such bacteria metabolize fermentable carbohydrates, acids are produced as a byproduct that lower the pH value of oral and plaque fluids, therefore creating an acidic oral environment. The bacterial acids (including particularly lactic acid) may then, among other things, leach minerals out of tooth structures within the oral cavity. Some of the most harmful caries-causing bacteria, such as mutans streptococci for example, produce such acids and are able to live and thrive in acidic environments.

Besides producing acids, some bacteria may also produce water-insoluble carbohydrate polymers. These polymers allow the bacteria to firmly adhere to tooth surfaces and other structures while producing acids, therefore resulting in surfaces that are exposed to high concentrations of acids over long periods of time. Streptococcus mutans and Streptococcus sobrinus are infamous for their active role in acid production, water-insoluble carbohydrate polymer production, and caries development. Other non-limiting examples of bacteria with similar properties include lactobacilli, nonmutans streptococci, actinomyces, and Veillonella species. In this application, any oral bacterium or microorganism that has the ability to ferment sugar to produce acids by metabolism and/or produce insoluble carbohydrate polymers, may cause or promote the development of dental caries.

As already mentioned, acids within the oral cavity cause the pH balance of the surrounding biofilm and environment to drop sometimes below critical levels (usually 5.7 or below), thereby initiating demineralization of tooth structures and causing such to be hypomineralized (i.e., lacking sufficient mineral content). For example, when enamel is demineralized, the acids permeate and dissolve the hydroxyapatite structure through gaps between enamel rods which are filled with water. A loss of calcium, phosphate, and other ions from the enamel tissue may occur and develop into an incipient carious lesion(s). With repeated acid attacks, caused by the further metabolism of fermentable carbohydrates by bacteria, the lesion(s) may expand further into the dentin and pulp chamber(s).

Another example of demineralization occurs on root surfaces. Root surfaces are covered by a thin protective layer of cementum. When gingival recession occurs causing cementum surfaces below the cemento-enamel junction to be exposed, such surfaces are susceptible to harmful acid attacks when pH levels of the oral environment are critically low. Acid exposure may cause, among other things, the removal of calcium, phosphate, and other ions from the tooth to be lost, causing hypomineralization. Eventually with repeated attacks, carious lesions may be created that are capable of expanding into other tooth structures such as the dentin and pulp chamber(s) if not treated. Other conditions, such as root sensitivity for example, may also occur if such surfaces are hypomineralized or exposed to harmful acids.

Hypomineralization of the enamel, cementum, or dentin may also exist or occur by other means besides demineralization. Non-limiting examples of naturally occurring hypomineralized areas may include amelogenesis imperfecta, dentinogenesis imperfecta, enamel hypoplastic lesions, etc.

Hypomineralized areas, including those found naturally, caused by demineralization, as well as carious lesions, may be remineralized. Remineralization is the restoration of minerals to tissues and structures. Natural remineralization involves, in part, the flow of saliva over teeth, biofilm, and other surfaces throughout the oral cavity. When saliva is stimulated and present within the oral cavity, under normal conditions, it may act as a buffer to the acids and raise the pH of the oral environment to safer levels. If the pH is raised to an appropriate level (usually above 5.7, but more preferably between 6.5-8.5), calcium and phosphate ions naturally found within the saliva may restore lost minerals to hypomineralized areas and possibly even reverse carious lesions.

However, natural remineralization can be a very slow process and is not guaranteed to remineralize due to the common fluxation of the pH in the oral environment, mostly caused by new foods or drinks introduced into the oral cavity. Since it is very common for persons to eat throughout the day and even snack between meal times, it can be very difficult for the pH to stay above the critical level for sufficient time periods to allow remineralization to occur because of the continual production of acids by bacteria consuming fermentable carbohydrates. Therefore, natural remineralization is not as effective as desired and attempts have been made in the art to enhance the remineralization process.

One of the most widely known efforts to aid remineralization involves using fluorine. Many commercially available products and dentifrice compositions contain various forms of fluorine ions. However, there are some instances where fluorine-ion compositions may not be desired.

In view of the foregoing, there is an ongoing need in the art for a composition that mineralizes dental hard tissues in the oral cavity and reduces the development of caries-inducive microflora. There is a further need in the art for a composition containing increased levels of minerals that enhances natural remineralization with or without the use of fluorine.

### BRIEF SUMMARY OF THE INVENTION

The present invention relates generally to a composition having increased levels of minerals for mineralizing hard tissues within the oral cavity and an ability to reduce caries-inducive microflora. The composition also has, among other things, an anti-dental caries function that promotes beneficial oral health.

A first general aspect of the present invention provides for an oral composition having an ability to mineralize dental hard tissues and reduce caries-inducive microflora, wherein said composition comprises a noncariogenic carbohydrate, a mineral source containing one or more calcium compounds, magnesium compounds, phosphate compounds, or trace minerals, or combinations thereof, and a thickening agent.

A second general aspect of the present invention provides for an oral composition having an ability to mineralize dental hard tissues and reduce caries-inducive microflora, wherein said composition comprises xylitol, a mineral source containing one or more calcium compounds, magnesium compounds, phosphate compounds, or trace minerals, or combinations thereof, and a thickening agent.

A third general aspect of the present invention provides for an oral composition having an ability to mineralize dental hard tissues and reduce caries-inducive microflora, wherein said composition comprises xylitol, calcium glycerol phosphate, magnesium glycerol phosphate, calcium lactate gluconate, one or more trace mineral compounds, and a thickening agent.

### DETAILED DESCRIPTION OF THE PREFERRED EMBODIMENTS

I. Introduction

The present invention relates to an oral composition having increased levels of minerals for mineralizing dental hard tissues within the oral cavity and an ability to reduce caries-inducive microflora. The composition also has, among other things, an anti-dental caries function that promotes beneficial oral health.

To provide context for interpreting the scope of the invention, certain terms used throughout the application will now be defined.

The term, “hypomineralization” refers to dental hard tissues in the oral cavity that are lacking optimal mineral content. Hypomineralization may occur by many means, but particularly by formation abnormalities, demineralization processes caused by acidic pH levels, and carious lesions.

As used herein, the term “mineralization” refers to the introduction of or restoration of minerals to dental hard tissues within the oral cavity. The term not only includes remineralizing areas that are hypomineralized, but may also include mineralizing and strengthening tissues that may be healthy, existing, and/or newly erupted.

The term “tissues” used herein refers to dental hard tissues within the oral cavity. “Dental hard tissues” may include the crown and root structure of a tooth, specifically enamel, dentin, and cementum.

“Anti-dental caries function(s)” used herein refers to functions of preventing and/or treating dental caries. The term can refer to one or more of the following properties: 1) a pH buffering ability to prevent pH reduction from acids introduced to the oral cavity or produced by oral microflora; 2) an ability to reduce or favorably modify caries-inducive microflora; 3) an ability to prevent microflora from producing insoluble carbohydrate polymers and other byproducts; 4) an ability to have chaotropic biofilm properties, and 5) an ability to promote mineralization of dental hard tissues within the oral cavity.

The term “noncariogenic carbohydrate” refers to a carbohydrate that has an anti-dental caries function with one or more of the above listed properties. The term may further refer to a carbohydrate that does not include fermentable sugars or other components that are prone to causing dental caries. As non-limiting examples, a noncariogenic carbohydrate may include carbohydrates such as xylitol, erythritol, maltitol, maltitol syrups, lactitol, isomalt, other polyols, tagatose, trehalose, isomaltulose, cellulose, modified cellulose, or polydextrose, or mixtures thereof. However, within this application, any carbohydrate that is capable of providing an anti-dental caries function may qualify as a suitable noncariogenic carbohydrate depending on its interaction properties with other ingredients described in this application.

II. Exemplary Compositions

The present invention relates generally to a composition having an ability to mineralize dental hard tissues within the oral cavity and reduce caries-inducive microflora. The composition also has, among other things, an anti-dental caries function that promotes beneficial oral health.

In an embodiment of this invention, the composition comprises a noncariogenic carbohydrate. The noncariogenic carbohydrate is a component of the present invention that provides one or more anti-dental caries functions. The noncariogenic carbohydrate may also provide additional functions within the present invention, such as acting as a humectant, sweetener, carrier for various substances or elements, salivary stimulant, ect. As mentioned previously, a noncariogenic carbohydrate may include xylitol, erythritol, maltitol, maltitol syrups, lactitol, isomalt, other polyols, tagatose, trehalose, isomaltulose, cellulose, modified cellulose, or polydextrose, or combinations thereof.

In an embodiment, xylitol may be used as a noncariogenic carbohydrate. Xylitol, a five-carbon sugar alcohol, is essentially a non-fermentable carbohydrate under conditions prevailing in the human oral cavity. As a result, xylitol may provide anti-dental caries functions, as well as other functions within the present invention. For example, xylitol may help prevent dental caries by functioning as an indirect buffering agent. Xylitol's naturally sweet flavor stimulates the salivary glands, therefore producing saliva that naturally contains higher levels of minerals. Such mineral-rich saliva may aid in buffering the effects of an acidic environment and increase the pH of the oral cavity to safer levels. Xylitol may also act as a carrier for supplemental mineral sources that are capable of buffering and increasing the pH of the oral environment above critical levels.

Xylitol may also prevent dental caries by functioning as an indirect mineralization agent. Xylitol may promote natural mineralization by stimulating and increasing saliva flow, therefore causing saliva to be produced that contains increased levels of calcium and phosphate ions that may mineralize tissues and structures within the oral cavity. Xylitol may also act as a carrier for supplemental mineral sources that enhance mineralization.

Another function of xylitol is that it may reduce and/or favorably modify caries-inducive microflora, as well as serve as an anti-microbial of such in certain circumstances. Most bacteria in the oral cavity that are responsible for causing dental caries metabolize fermentable carbohydrates to grow and develop. As such bacteria ingest xylitol (an essentially non-fermentable carbohhydrate under conditions prevailing in the human oral cavity) in consistent quantities, the bacteria may be unable to ferment the xylitol and further develop or carry out necessary functions. The bacteria's ingestion of xylitol may prevent the uptake of other essential fermentable carbohydrates sources, consequently weakening the bacteria and gradually depleting it of its energy sources. Such wastes of energy (by consuming xylitol and the inhibition of fermentation) may weaken the bacteria to the extent that its productions and byproducts (i.e., insoluble carbohydrate polymers, harmful acids, etc.) are affected. Upon such circumstances, the bacteria may decrease their production of harmful acids and lose their ability to adhesively bond to surfaces within the oral cavity. Over time, as the bacteria ingest and accumulate significant levels of xylitol, the xylitol metabolites may become toxic to some of these pathogenic bacteria.

As previously mentioned, xylitol also may function naturally as a sweetener, saliva stimulant, and/or humectant. Xylitol may also serve as a carrier for various ingredients within the present invention.

In an embodiment of this invention, the composition may also comprise a mineral source containing one or more mineral compounds with anti-dental caries functions. The mineral source contains mineral compounds that may enhance the natural mineralization process and aid in buffering the effects of harmful acids within the oral cavity. The mineral source may also aid in increasing the pH of the oral environment. In an embodiment, the mineral source may include one or more soluble calcium compounds, magnesium compounds, phosphate compounds, or trace mineral compounds, or combinations thereof.

Non-limiting examples of soluble calcium compounds may include calcium glycerol phosphate, calcium lactate gluconate, calcium fructo-borate, calcium hydrogen phosphate, “amorphous” calcium phosphate, or calcium phosphopeptides, or combinations thereof. The present invention is also intended to include any other calcium source that is capable of promoting mineralization and beneficial oral health

Non-limiting examples of magnesium compounds may include magnesium glycerol phosphate or magnesium boroascorbate, or combinations thereof. The present invention is also intended to include any other magnesium source that is capable of promoting mineralization and beneficial oral health

Non-limiting examples of phosphate compounds may include calcium hydrogen phosphate, amorphous calcium phosphate, calcium glycerol phosphate, or calcium phosphopeptides, or combinations thereof. The present invention is also intended to include any other phosphate source that is capable of promoting mineralization and beneficial oral health

Non-limiting examples of trace mineral compounds may include strontium, boron, selenium, or fluorine, or combinations thereof may be used. Although fluorine has been proven to be effective in aiding mineralization processes, the present invention embodies compositions that effectively mineralize hard tissue surfaces with or without the use of fluorine.

In an embodiment of this invention, the composition may also comprise a thickening agent. A thickening agent may be used to thicken the mixture into a gel, slurry, paste, or any other consistency as desired. Non-limiting examples of suitable thickeners may include glycerin, carboxy methylcellulose, microcrystalline cellulose, hydroxypropyl methylcellulose, xanthan gum, Z-Trim®, or vegetable gum such as carob, guar, and gum arabic. Glycerin is a sugar alcohol that besides acting as a thickener, may also act as a natural liquid carrier and/or preservative. In another embodiment, where an alternative thickener is used such as carboxy methylcellulose, the addition of water and/or a preservative may be required.

In an embodiment of this invention, the composition may also require a preservative. In an embodiment, the preservative may include benzoates, sorbates, grapefruit seed extract, colloidal silver, or cetylpyridinium chloride, or combinations thereof.

In an embodiment of this invention, the composition may also include additives such as, but not limited to, nutritional cofactors, botanicals, enzymes, amino acids, immunoglobulins, flavoring, artificial sweeteners, malodor control, or water, or combinations thereof. Non-limiting examples of nutritional cofactors may include vitamins such as ascorbic acid (Vitamin C), folic acid, carotene, cholecalciferol (Vitamin D), phytonadione (Vitamin K), B vitamins, or any other vitamin that is proven or known in the art to promote beneficial oral health. Non-limiting examples of botanicals may include aloe vera, cranberry extract, cinnamon, green tea extract, Applephenon™, pomegranate extract, coffee berry extract, cocoa, cacao bean husk extract, or any other botanical that is proven or known in the art to promote beneficial oral health. Non-limiting examples of enzymes may include lactoferrin, hexose oxidase, coenzyme Q10 or any other enzyme that is proven or known in the art to promote beneficial oral health. Non-limiting examples of amino acids may include arginine, lysine, proline, casein phosphopeptide, various caseinates, or any other amino acid or peptide derivative that is proven or known in the art to promote beneficial oral health. Non-limiting examples of immunoglobulins may include IgG and IgA or any other immunoglobulin that is proven or known in the art to promote beneficial oral health. Non-limiting examples of flavoring may include peppermint, spearmint, cinnamon, or any other flavoring that is proven or known in the art to promote benificial oral health. Non limiting examples of artificial sweeteners may include aspartame, sucralose, acesulfame K, or neotame or any other sweetener that is proven or known in the art to promote beneficial oral health. Non-limiting examples of malodor controllers may include Applephenon™, zinc citrate, or any other malodor controller that is proven or known in the art to promote beneficial oral health.

The following are several examples of compositions that have been formulated according to the present invention. Such exemplary formulations are given by way of example, and not by limitation. Unless otherwise indicated, all percentages are by weight.

### EXAMPLE 1

A composition having an anti-dental caries function in the form of a gel was formed by a commonly used method and comprised the following components:

| | |
|---|---|
| Water | 83% |
| Xylitol | 14% |
| Calcium glycerol phosphate | 1.5% |
| Carboxy methylcellulose | 1.25% |
| Grapefruit seed extract | 0.25% |

### EXAMPLE 3

A slurry composition having an anti-dental caries function in the form of a paste was formed by a commonly used method and comprised the following components:

| | |
|---|---|
| Water | 76% |
| Xylitol | 12% |
| Tagatose | 3% |
| Calcium glycerol phosphate | 2% |
| Calcium lactate gluconate | 1.5% |
| Xanthan gum | 2% |
| Cranberry extract | 1.25% |
| Magnesium glycerol phosphate | 1% |
| Peppermint flavor | 0.75% |
| Applephenon ™ | 0.25% |
| Grapefruit seed extract | 0.25% |

### EXAMPLE 4

A liquid composition having an anti-dental caries and breath-freshening function was formed by a commonly used method and comprised the following components:

| | |
|---|---|
| Water | 82% |
| Xylitol | 15% |
| Calcium glycerol phosphate | 1.25% |
| Peppermint flavor | 1.25% |
| Applephenon ® | 0.25% |
| Grapefruit seed extract | 0.25% |

### EXAMPLE 5

A dry composition having an anti-dental caries function in the form of a powder was formed by a commonly used method and comprised the following components:

| | |
|---|---|
| Xylitol | 70% |
| Calcium lactate gluconate | 10% |
| Calcium glycerol phosphate | 8.5% |
| Xanthan gum | 8.5% |
| Magnesium glycerol phosphate | 1.5% |
| Peppermint flavor | 1.5% |

III. Exemplary Methods of Use

As discussed, the present invention may be prepared and used in a variety of forms. For example, in one embodiment, the composition may be a premixed liquefied mixture, such as but not limited to a rinse, spray, gel, paste, slurry, etc. Non-limiting examples of application of these embodiments may include a rinsing solution, oral spray, oral gel, prophy paste, etc. In another embodiment, the composition may be in a powder form that may later be mixed with liquid, such as water for example, to a desired consistency. Non-limiting examples of application of this embodiment may include a powder that is capable of being mixed at home or at a dental office, a powder composition that is used in conjunction with an oral irrigating device, prophy jet, etc. In another embodiment, the composition may be a liquid that includes a foaming agent, therefore being able to be used as foam if desired. In an alternative embodiment, the composition may be contained within an edible sponge-carrying medium. As mentioned above, these examples are not to be limiting in any context and the present invention includes all mediums that are capable of delivering the composition to tissues throughout the oral cavity, for mineralization of hard tissues and reduction of caries-inducive microflora.

One example in which the present invention has been found to be particularly effective is in the treatment of decalcification scarring or “white spots;” the decalcification scarring being generalized or localized within the oral cavity. Such conditions are commonly found in patients receiving orthodontic treatment, perhaps due to the difficulty of some patients in complying adequately with homecare regimes. The present invention is useful in treating such areas by mineralizing, strengthening, and even possibly reversing the harm. In some instances, decalcification scarring has found to be drastically diminished or even completely eliminated due to treatment of the present invention. To treat the harmed areas, the practitioner may etch the area (by chemical etching, mechanical etching, or both) and then apply the composition to the area, supplying the hypomineralized area with needed minerals. The present invention has also been found to be effective as a preventive measure for decalcification scarring and is therefore helpful for patients to utilize during orthodontic, during other treatment, or on a routine basis.

The present invention may also be very useful in treating amelogenesis imperfecta, dentinogenesis imperfecta, or other enamel hypoplastic lesions in which areas of tooth structure are underdeveloped or hypomineralized. The present invention may help mineralize such weakened areas and may also possibly even improve scarring from such underdevelopment or hypomineralization.

The present invention may also be effective in decreasing sensitivity, whether caused by exposed cementum or by carious lesions for example. If sensitivity is due to cementum exposure, the composition may mineralize and strengthen the cementum, therefore decreasing exposed root surface sensitivity. If the sensitivity is due to carious lesions, the present invention may also mineralize and reverse incipient to advanced carious lesions, therefore strengthening the tooth structure, decreasing sensitivity, and possibly even preventing future invasive treatment.

The present invention may also aid in treating patients with xerostomia, Sjögren's Syndrome or other conditions that cause dryness in the mouth due to the absence of or diminished quantity of saliva. The lack of saliva and the resulting dry condition of the mouth are important factors that can lead to oral discomfort and disease, such as carious lesions and/or periodontal disease. The present invention may stimulate salivary glands and help increase the production of mineral-rich saliva, therefore alleviating oral discomfort and helping to prevent diseases from occurring or further developing. In patients suffering from permanently reduced salivary production, such as patients undergoing radiation or other cancer therapy, the present invention may also provide supplemental mineralization treatment besides increased saliva production. The supplemental mineralization properties of the present invention allows the composition to effectively buffer acidic effects in the oral cavity and raise the pH to safer levels so as to prevent or stop demineralization from occurring.

The proposed invention may also be used to reduce and/or favorably modify caries-inducive microflora. As the bacteria ingest the present invention in consistent quantities, the bacteria may become unable to ferment needed carbohydrate sources in order to develop or carry out necessary functions. Consequently, the bacteria may be weakened and its energy sources gradually depleted. Such wastes of energy (by consuming the present invention and the inhibition of fermentation) may weaken the bacteria to the extent that its productions and byproducts (i.e., insoluble carbohydrate polymers, harmful acid, etc.) are affected. Upon such circumstances, the bacteria may decrease their production of harmful acids as well as lose their ability to adhesively bond to surfaces within the oral cavity. Over time, as the bacteria ingest and accumulate significant levels of the present invention, the metabolites may accumulate and become toxic to these pathogenic bacteria.

Because of the invention's ability to reduce some pathogenic microflora within the oral cavity as described above, the present invention may also be used to help decrease gingival inflammation. When caries-inducive bacteria metabolize fermentable carbohydrates and produce harmful acids and other byproducts within the oral cavity, such productions may irritate and cause inflammation in gingival tissues. The present invention is able to help decrease such inflammation by reducing such microflora as previously described above, and by reducing the production of some harmful and irritating acids/byproducts.

It will also be appreciated that the present claimed invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. An oral composition having an ability to mineralize dental hard tissues and reduce caries inducive microflora, wherein said composition comprises:
a. a noncariogenic carbohydrate,
b. a mineral source containing one or more calcium compounds, magnesium compounds, phosphate compounds, or trace minerals, or combinations thereof, and
c. a thickening agent.

2. A composition in accordance with claim 1, wherein the noncariogenic carbohydrate includes xylitol, erythritol, maltitol, maltitol syrups, lactitol, isomalt, other polyols, tagatose, trehalose, isomaltulose, or polydextrose, or combinations thereof.

3. A composition in accordance with claim 1, wherein the calcium compound includes calcium glycerol phosphate, calcium lactate gluconate, calcium fructoborate, calcium hydrogen phosphate, calcium casein phosphopeptides, or amorphous calcium phosphate, or combinations thereof.

4. A composition in accordance with claim 1, wherein the magnesium compound includes magnesium glycerol phosphate or magnesium boroascorbate, or combinations thereof.

5. A composition in accordance with claim 1, wherein the phosphate compound includes calcium hydrogen phosphate, amorphous calcium phosphate, calcium glycerol phosphate, or calcium phosphopeptides, or combinations thereof.

6. A composition in accordance with claim 1, wherein the trace mineral compound includes strontium, boron, selenium, or fluorine, or combinations thereof.

7. A composition in accordance with claim 1, wherein the thickening agent includes cellulose, carboxy methylcellulose, modified cellulose, xanthan gum, vegetable gum, microcrystalline cellulose, hydroxypropyl methylcellulose, Z-Trim®, or glycerin, or combinations thereof.

8. A composition in accordance with claim 1, wherein the composition may additionally include nutritional cofactors, botanicals, enzymes, amino acids, flavoring, artificial sweeteners, malodor controllers, preservatives, or water, or combinations thereof.

9. A composition in accordance with claim 1, wherein the composition is sold in liquid or powder form.

10. An oral composition having an ability to mineralize dental hard tissues and reduce caries inducive microflora, wherein said composition comprises:
a. xylitol,
b. a mineral source containing one or more calcium compounds, magnesium compounds, phosphate compounds, or trace minerals, or combinations thereof, and
c. a thickening agent.

11. A composition in accordance with claim 9, wherein the calcium compound includes calcium glycerol phosphate, calcium lactate gluconate, calcium hydrogen phosphate, or calcium fructoborate, or combinations thereof.

12. A composition in accordance with claim 9, wherein the magnesium compound includes magnesium glycerol phosphate or magnesium boroascorbate, or combinations thereof.

13. A composition in accordance with claim 9, wherein the phosphate compound includes calcium hydrogen phosphate, amorphous calcium phosphate, calcium glycerol phosphate, or calcium phosphopeptides, or combinations thereof.

14. A composition in accordance with claim 9, wherein the trace mineral compound includes strontium, boron, selenium, or fluorine, or combinations thereof.

15. A composition in accordance with claim 9, wherein the thickening agent includes carboxy methylcellulose, xanthan gum, vegetable gum, microcrystalline cellulose, hydroxypropyl methylcellulose, Z-Trim®, or glycerin, or combinations thereof.

16. A composition in accordance with claim 9, wherein the composition may additionally include nutritional cofactors, botanicals, enzymes, amino acids, flavoring, artificial sweeteners, malodor controllers, preservatives, or water, or combinations thereof.

17. A composition in accordance with claim 9, wherein the composition is sold in liquid or powder form.

18. An oral composition having an ability to mineralize dental hard tissues and reduce caries inducive microflora, wherein said composition comprises:
a. xylitol,
b. calcium glycerol phosphate,
c. magnesium glycerol phosphate,
d. calcium lactate gluconate,
e. one or more trace mineral compounds, and
f. a thickening agent.

19. A composition in accordance with claim 17, wherein the trace mineral compounds include strontium, boron, selenium, or fluorine, or combinations thereof.

20. A composition in accordance with claim 17, wherein the thickening agent includes cellulose, modified cellulose, carboxy methylcellulose, hydroxypropyl methylcellulose, xanthan gum, vegetable gum, or glycerin or combinations thereof.

21. A composition in accordance with claim 17, wherein the composition may additionally include nutritional cofactors, botanicals, enzymes, amino acids, flavoring, artificial sweeteners, malodor controllers, preservatives, or water or combinations thereof.

22. A composition in accordance with claim 17, wherein the composition is sold in liquid or powder form.
